(19) United States
(12) Patent Application Publication
(10) Pub. No.: US 2013/0090710 A1
(43) Pub. Date: Apr. 11, 2013

(54) ALLEVIATIVE PILLOW

(71) Applicant: Claudia Rimoli, Davie, FL (US)

(72) Inventor: Claudia Rimoli, Davie, FL (US)

(21) Appl. No.: 13/703,026

(22) PCT Filed: Jun. 17, 2011
(86) PCT No.: PCT/US2011/040997

§ 371 Date: Dec. 9, 2012

Related U.S. Application Data
(60) Provisional application No. 61/356,336, filed on Jun. 18, 2010.

(51) Int. Cl. A61F 7/10; A61F 7/08

(52) U.S. Cl. CPC A61F 7/10; A61F 7/08
USPC 607/108; 607/112

(57) ABSTRACT

A back alleviative pillow uses hot and cold packs combined with memory foam in a pillow to provide comfort and alleviation from pain to an operator. The pillow includes a plurality of adjustable straps such that the pillow may be positioned at a desired location on the operator's body. The straps may secure the pillow to the operator, to a chair back, or other structure proximal to the desired point of contact on an operator's body.

### CROSS-REFERENCE TO RELATED APPLICATIONS

This patent application is the national phase under 35 U.S.C. §371 of PCT International Application No PCT/US2011/040997 which claims priority to U.S. Provisional Patent Application No. 61/356,336 filed on Jun. 18, 2010.

### BACKGROUND

1. Field

The present invention relates to a therapeutic device and specifically to an alleviative pillow may be worn on a person, attached to a chair or otherwise placed in a plurality of ergonomic positions to improve comfort and health of the user.

2. Background

A large portion of the adult population experiences discomfort ranging from minor aches to chronic and persistent pain, often in the back. According to the National Institutes of Health (NIH), back pain affects eight out of 10 people at some point in their lives and is the most common cause of activity limitation for people under 45.

Surgery is normally a last option, and often a desperate measure, all but a small percentage of sufferers elect other methods with which to live with the discomfort. Billions of dollars are spent every year on medical visits, physical therapy, aids such as lumbar pillows, and in many instances, surgery. Lumbar spine pillows, as well as other heating/cold options, top the list of products subscribed by medical authorities to provide relief for back pain and discomfort.

The market for lumbar support pillows is vast and developed, but fractured, with many types of inner “elements”, ranging from gel to seeds. There have been many different products over time, which were developed to relieve aches and pains. Electric heating pads and ice packs have been the norm for many years. Electric pads are not particularly comfortable, and are not mobile. They expend energy while in use, and are limited to home use, typically. Ice packs require a waterproof aspect, to be affective, and normally are quick to thaw, melt, and lose their efficacy.

Seeds and other “green” products are on the market, and these products often break down, rot, or deteriorate within a few months of regular usage.

There are a variety of means to provide comfort and/or support to an operator, as may be seen in the following patents:

U.S. Pat. No. 6,419,652 for a Back belt and method; U.S. Pat. No. 6,099,645 for a Vertical semiconductor wafer carrier with slats; U.S. Pat. No. 5,697,619 for a Radial seal for air preheater; RE34,883 for a Simplified orthopaedic back support U.S. Pat. No. 5,378,225 for a Heated back support; U.S. Pat. No. 5,062,414 for a Simplified orthopaedic back support; U.S. Pat. No. 4,993,409 for a Back support; U.S. Pat. No. 4,966,145 for an Automatic body temperature adjuster; U.S. Pat. No. 4,195,640 for a Lower torso support appliance; and U.S. Pat. No. 4,108,149 for a Maternity brace.

However, all of these designs have their own drawbacks. It is desirable to provide a pillow support device that may be easily adjusted to properly fit and individual and may be secured in a variety of positions.

It is therefore desirable to provide Superior Back Comfort, Back and Posture Support, Pain Free Commutes; Revitalize While You Drive, FDA Approved Heating Element, Temperature Sensitive Memory Foam, Therapeutic Relief for Aches and Pains, Molds Naturally to your Body, Hypo-Allergenic Memory Foam, Fire Retardant Memory Foam, Long-lasting Heating and Cooling Comfort, Reduces Lower Back Pain and Tension, Convenient for Home, Work, Commute and Travel, Safe and Easy to Use, No Batteries, Chargers or Plug-Ins.

### BRIEF DESCRIPTION OF THE DRAWINGS

FIG. 1 is a perspective view of a backpack in accordance with the principles of the invention;

FIG. 2 is a perspective view of a pillow in accordance with the principles of the invention having a cut-away view of one corner thus displaying the interior.

FIG. 3 is a top view of an alternative embodiment of a pillow in accordance with the principles of the invention;

FIG. 4 is a side view of an alternative embodiment of a backpack in accordance with the principles of the invention;

FIG. 5 is a top view of an alternative embodiment of a pillow in accordance with the principles of the invention;

FIG. 6 is a side view of an alternative embodiment of a backpack in accordance with the principles of the invention;

### SUMMARY OF THE INVENTION

The alleviative pillow may be ideal for work, travel and play, at home, the office, on the road or in the air. It may provide hours of temperature-enhanced relief and support for tired, aching upper and lower backs with heating and cooling elements.

If you sit for long periods of time your back needs extra support. The alleviative pillow of the invention can be used vertically or horizontally for extra back comfort and support in a home, office, car or plane.

The alleviative pillow may be ergonomically designed and may use temperature sensitive memory foam for the perfect fit. It molds to the upper or lower back, shoulders, legs or other regions of the body to which it may be applied and may provide comfortable and firm support to maintain balanced spinal alignment. Its support may relieve pressure on important nerves and muscles in your back that can become inflamed and painful. The alleviative pillow may help to relieve back pain, muscle soreness and stiffness as well as alleviate the pain associated with back injury and chronic conditions such as sciatica, scoliosis, arthritis, fibromyalgia, multiple sclerosis and muscular dystrophy.

It is therefore an object of the invention to provide comfort and posture support, easy driving for long periods and to apply heat and/or cooling to a person with a durable, reliable, light weight, multifunctional device.

There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### DETAILED DESCRIPTION OF THE INVENTION

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

The alleviative pillow of the invention may help relieve upper and/or lower back pain, and may make sitting for long periods of time more comfortable for a user. It may be adjustable to suit individual needs. An adjustable strap may secure the pillow to almost any chair or seat with ease. Traveling, or sitting at a desk all day, may be an uncomfortable experience. Sitting for long periods of time may be more comfortable when an alleviative pillow of the invention is used.

The alleviative pillow may include a backpack. By wearing the backpack, a user may avoid being uncomfortable in a chair or when sitting. A user simply uses the straps to wear the backpack, adjusting it such that the alleviative pillow is positioned in a desired location such as for example adjacent to the lumbar region. The alleviative pillow may keep the user warm in the winter and cool in the summer through the use of hot/cold packs that may be inserted into the pillow. The backpack may optionally be strapped to a chair instead of directly to a user.

### How it Works:

The human back is designed in the shape of an S. But when we sit, especially for extended periods of time, the muscles alongside the upper back may relax and sag, forcing their weight to “sit” on your waist and lower back. The result may be lower back pain and stiffness, and eventually, compression of the discs in the lower back. The alleviative pillow of the invention may redistribute muscle weight evenly along the entire spinal column, realigning the body so that it works naturally and efficiently. By helping maintain ideal posture, the alleviative pillow may alleviate accumulated stress in the lower back and may reduce or eliminate the source of strain. By correcting sitting posture, the alleviative pillow of the invention may work to restore the spine to its proper alignment. Working at a desk for long hours, riding in a car or plane, even watching a movie or play may be comfortable and enjoyable when the alleviative pillow is used. Resting after a workout may no longer causes stiffness and soreness in the lower back.

Clinicians have long believed that the application of both heat and cold may have significant therapeutic effects on sore, strained muscles. Depending on the situation and a user's preferences, either therapy may provide pain relief, muscle relaxation, decreased muscle spasms, and reduced sensitivity. Applying warmth to the lower back may also promote comfort and relaxation, while cooling can make you feel invigorated and refreshed.

An alleviative pillow in accordance with the principles of the present invention may includes a cushion encased in a sleeve. The sleeve may include one or more pockets on opposite sides for accommodating one or more hot/cold packs. Once the packs are inserted in to their pockets, the pillow, sleeve and packs may be slid into a backpack. The backpack may include two or more straps. As may be seen in the drawings, these straps may interact to provide securement of the invention to an operator. In addition to being attachable directly to the operator, the backpack may also be attached to a car seat, desk chair or other object. The straps may further allow the backpack to be worn as a lumbar support. In addition to support, the use of a hot/cold pack may provide comfort during extended periods in hot or cold weather.

Referring now to FIG. 1, an alleviative pillow system 10 in accordance with the principles of the invention may include a backpack 14 having a zipper 20 that provides access to an interior compartment into which an alleviative pillow and other items may be placed. Backpack 14 may be comprised of any suitable material, including microfiber, synthetic polymers, wool, cotton, and the like, that may be thermally conductive. A strap 16 may be attached at the lower left corner 15 and may extend the length of the backpack to attach to upper left corner 13. Strap 16 may include one or more adjusting buckles 26 that may allow the length of the strap 16 to be adjusted. Similarly, strap 18 may be attached to a lower right corner 11 and extend to an upper right corner 21 and include adjusting buckles 28 that may allow the length of the strap 18 to be adjusted.

Also shown in FIG. 1 is a multifunction strap 12. Multifunction strap 12 may include an adjusting buckle 24 to adjust its length. Snap hooks 22 are located on each end of multifunction strap 12 and allow it to connect to glides 17 and 19 on straps 16 and 18 respectively. In use, straps 16 and 18 may be placed about a user's shoulders in the manner of a backpack and multifunction strap 12 may be wrapped around an operator's waist so as to hold the body 14 securely against the back or front of a wearer. The straps 16, 18 and 20 may be adjusted to lengths that secure the backpack 14 at a desired location in relation to the user's body.

In this embodiment, the straps 16 and 18 are permanently affixed to the backpack. The multifunctional strap 12 may be connected to one or both of the gliders.

FIG. 2 shows a cut-away view of a pillow 30 of an alternative embodiment of the a backpack in accordance with the principles of the invention. Backpack 30 has an integument 32 made of cotton or similar material that may absorb moisture but allow heat and/or cold to pass through it. Backpack 30 has three short attachment straps 34 each having a D-ring 36. These attachment straps 34 and D-rings 36 allow one or more multifunction straps, such as multifunctional strap 12 of FIG. 1, to be attached to the pillow 30 directly to facilitate better and accurate placement of the backpack 30 on a desired location on a user's body, or on a chair or other furniture.

Backpack 30 includes a zipper 38 that allows access to its interior, which contains a memory foam pad 40 or the like and/or a hot/cold pack 44. The hot/cold pack 44 may contain a flexible element that may be cooled or warmed in a freezer or microwave and used to transfer heat or cold for extended periods. Optionally, the hot/cold pack 44 may be battery operated.

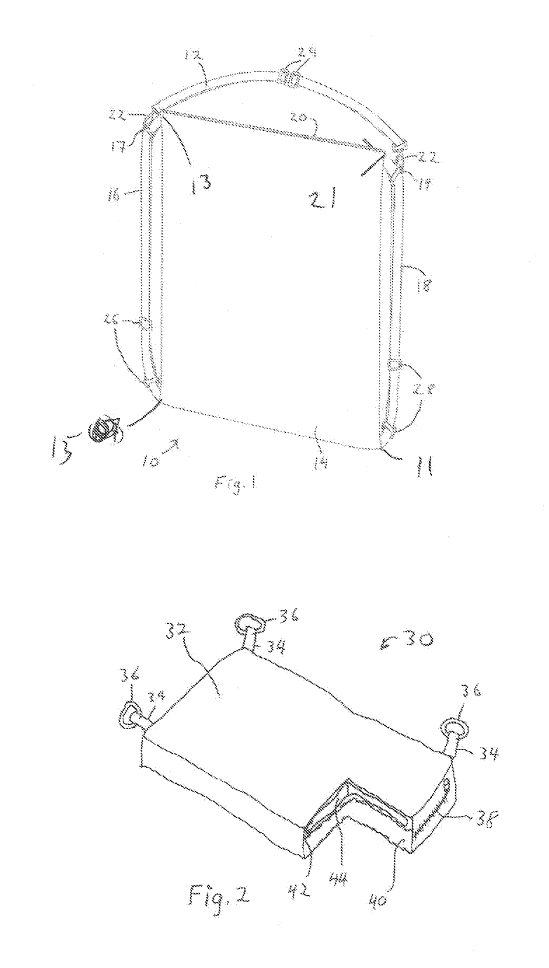

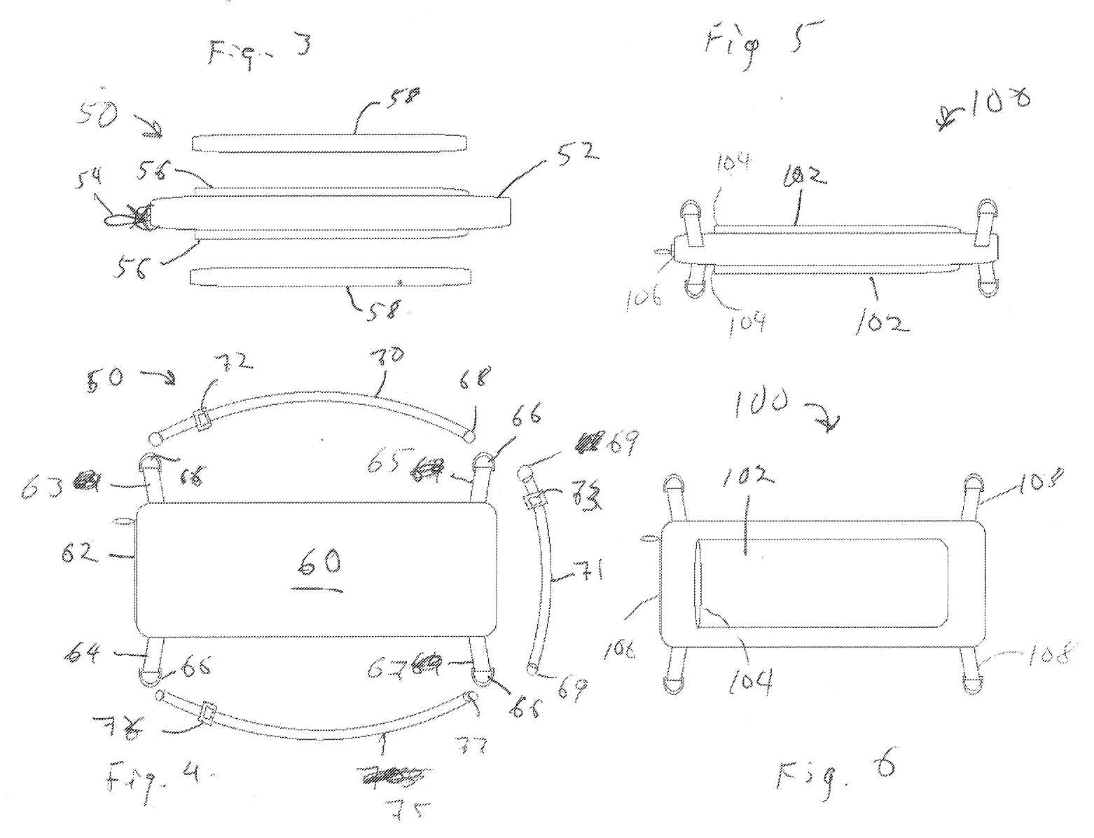

FIGS. 3 and 4 show an alternative embodiment of an alleviative pillow 50 in accordance with the principles of the present invention. FIG. 3 shows a top view of an alleviative pillow 50 that may include a pillow 52 comprised of any suitable pillow material stuffed with any suitable pillow stuffing, for example memory foam, goose down and the like. Pillow 52 optionally may include an opening 54 to allow removal of the stuffing material such that the pillow case may be washed. Pillow 52 may include two pockets 56 for holding hot/cold packs 58 on opposite sides of the pillow 52. The hot/cold packs 58 may be of a design that allows the packs to be cooled, for example in a freezer, or alternatively heated, for example in a microwave, such that they are at a desired temperature and remain at that temperature for an extended period of time. It may be desirable to provide one of the hot/cold packs 56 at a hot temperature and the opposite hot/cold pack 56 at a cold temperature. This may allow a user to rapidly switch from applying hot to cold temperature to a desired location by simply flipping over the alleviative pillow. The pockets 56 may be open ended or may optionally be sealed closed by means of buttons, zippers, elastic or other mechanisms to more securely retain within them the hot/cold packs 58.

FIG. 4 shows a backpack 60 which has an interior compartment into which a pillow 52 may be inserted through opening 62 having a zipper or other sealing mechanism. Backpack 60 may also include four attachment straps 63, 64, 65 and 67 each having a D-ring 66. Snap hooks 68 may be located on one or more multifunctional strap 70 and may allow the multifunctional strap to be attached to any of the D-rings 66. Straps 70 may include adjustment buckles 72 to facilitate adjustment of the length of the straps. Also shown is multifunctional strap 71 having adjustment buckle 73 and snap hooks 69, and multifunctional strap 75 having adjustment buckle 73 and snap hooks 77.

The invention is capable of several configurations. One or more multifunctional straps 70 and 71 may be connected to one or more attachment straps and placed over the headrest of a car seat such that the alleviative pillow hangs down from the headrest allowing a user's back to rest against the alleviative pillow. For example, multifunctional strap 71 may be attached to attachment straps 65 and 67 such that the alleviative pillow hangs on the back of a chair so that its width is less than the height.

Alternatively, multifunctional strap 70 may be attached to attachment straps 63 and 65 and the alleviative pillow may be attached to a computer chair such that its width is greater than its height. It may be desirable to attach a strap to attachment straps 64 and 67 to better secure the alleviative pillow to the chair back. The straps may be adjusted to tightly fit about the chair back to secure the alleviative pillow in a desired location on the chair back. For example, it may be positioned at the lower region of the back of the chair to provide lumbar support. This allows a user to sit for long periods of time now with no discomfort.

Alternatively, the multifunctional straps may criss-cross, for example with multifunctional strap 70 attached to attachment straps 63 and 67 and multifunctional strap 75 attached to attachment straps 64 and 65.

In addition to placing the alleviative pillow in a chair or other furniture. It may be worn directly on a person's body like a backpack. This may allow a user to place the alleviative pillow against his or her back or front, shoulders, neck, arms, legs, lap, buttocks or other desired locations.

FIGS. 5 and 6 show another alternative embodiment of an alleviative pillow in accordance with the principles of the invention. An alleviative pillow 100 has two pockets 102 located on opposite sides and capable of retaining within them hot/cold packs which may be inserted through openings 104. Openings 104 may optionally include a sealing mechanism such as a zipper. The alleviative pillow 100 includes an opening 106 that may be opened and closed using a sealing mechanism such as a zipper. Rather than the having a pillow that is inserted within a backpack, in this embodiment, the alleviative pillow 100 has attachment straps 108 directly on it instead of a separate backpack into which a pillow and hot/cold packs may be placed.

In the embodiments shown in FIGS. 3-6, the alleviative pillows and backpacks are shown having a substantially rectangular shape. Optionally, the alleviative pillow and backpack may comprise any shape including round, ovular, polygonal or other shapes. Similarly, while the backpacks shown typically includes attachment straps at its corners, straps may be attached at any desirable location to facilitate securing the pillow to a desirable location on a person's body or furniture. The multifunctional straps may be attached to any attachment straps and adjusted to a desired length in order to facilitate proper placement of the alleviative pillow.

Those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

The invention claimed is:

1. An alleviative pillow comprising:
a pillow having one or more pockets;
one or more hot/cold packs capable of being placed within the one or more pockets;
a backpack having two or more attachment straps and an interior compartment into which the pillow may be placed; and,
one or more multifunctional straps removably attachable to the two or more attachments straps.

2. The alleviative pillow of claim 1 wherein the pillow has two pockets on opposite sides of the pillow and two hot/cold packs.

3. The alleviative pillow of claim 1 wherein the backpack has four attachment straps.

4. The alleviative pillow of claim 1 wherein the cavity of the backpack may be sealed by a zipper.

5. The alleviative pillow of claim 1 wherein the one or more multifunctional straps comprise three multifunctional straps each having an adjustable length.

6. The alleviative pillow of claim 2 wherein the backpack is rectangular and the two or more attachment straps comprise four attachment straps, one on each of the corners of the backpack;
wherein the one or more multifunctional straps comprise three multifunctional straps each having an adjustable length; and,
wherein the multifunctional straps are attachable to the four attachment straps by means of snap hooks.

7. An alleviative pillow comprising:
a pillow having one or more pockets;
one or more hot/cold packs capable of being placed within the one or more pockets;
two or more attachment straps; and,
one or more multifunctional straps removably attachable to the two or more attachments straps.

8. The alleviative pillow of claim 7 wherein the pillow has two pockets on opposite sides of the pillow and two hot/cold packs.

9. The alleviative pillow of claim 7 wherein the backpack has four attachment straps.

10. The alleviative pillow of claim 7 wherein the pillow has an opening that allows for placement of and removal from padding within an interior compartment of the pillow.

11. The alleviative pillow of claim 7 wherein the one or more multifunctional straps comprise three multifunctional straps each having an adjustable length.

12. The alleviative pillow of claim 8 wherein the pillow is rectangular and the two or more attachment straps comprise four attachment straps, one on each of the corners of the pillow;
wherein the one or more multifunctional straps comprise three multifunctional straps each having an adjustable length; and,

13. wherein the multifunctional straps are attachable to the four attachment straps by means of snap hooks.

14. An alleviative pillow comprising:
a pillow having one or more pockets;
one or more hot/cold packs capable of being placed within the one or more pockets;
a rectangular backpack having two integrated straps and an interior compartment into which the pillow may be placed; and,
a multifunctional strap removably attachable to the two integrated straps.

15. The alleviative pillow of claim 14 wherein the pillow has two pockets on opposite sides of the pillow and two hot/cold packs.

16. The alleviative pillow of claim 14 wherein the integrated straps each have one or more glides to which the multifunctional strap may removably attach.

17. The alleviative pillow of claim 14 wherein the cavity of the backpack may be sealed by a zipper.

18. The alleviative pillow of claim 14 wherein the lengths of the integrated straps and the multifunctional strap may be adjusted.

19. The alleviative pillow of claim 15 wherein the backpack is rectangular and the two integrated straps are each attached to two of the four corners of the backpack;
wherein the one or more multifunctional straps comprise three multifunctional straps each having an adjustable length; and,
wherein the multifunctional strap is attachable to the integrated straps by means of snap hooks.
